(19) United States
(12) Patent Application Publication
(10) Pub. No.: US 2024/0161921 A1
(43) Pub. Date: May 16, 2024

(54) BIOMETRIC INFORMATION PROCESSING DEVICE, INFORMATION PROCESSING SYSTEM, BIOMETRIC INFORMATION PROCESSING METHOD, AND RECORDING MEDIUM

(71) Applicant: NEC Corporation, Minato-ku, Tokyo (JP)

(72) Inventors: Kenichiro Fukushi, Tokyo (JP); Zhenwei Wang, Tokyo (JP); Chenhui Huang, Tokyo (JP); Fumiyuki Nihey, Tokyo (JP); Kentaro Nakahara, Tokyo (JP)

(73) Assignee: NEC Corporation, Minato-ku, Tokyo (JP)

(21) Appl. No.: 18/284,324

(22) PCT Filed: Apr. 1, 2021
(86) PCT No.: PCT/JP2021/014124

§ 371 Date: Sep. 27, 2023

(51) Int. Cl. G16H 40/67; A61B 5/00; A61B 5/11; G16H 50/30

(52) U.S. Cl. CPC G16H 40/67; A61B 5/112; A61B 5/6807; A61B 5/7203; G16H 50/30; A61B 2505/09

(57) ABSTRACT

A biometric information processing device includes an extraction unit that extracts a daily measurement value of a common measurement item common to facility measurement data measured in a facility from daily measurement data measured using the sensor data regarding movement of a foot of a user, and a correction unit that corrects a facility measurement value of the common measurement item stored in advance based on the extracted daily measurement value of the common measurement item.

### TECHNICAL FIELD

The present disclosure relates to a biometric information processing device or the like that processes sensor data measured with walking.

### BACKGROUND ART

With growing interest in healthcare that performs physical condition management, attention has been focused on a service that measures a gait including a walking feature and provides information according to the measured gait. Such a service requires a technique for accurately evaluating an exercise function in walking or the like. For example, an exercise function in walking or the like is evaluated in a facility such as a hospital or a sports gym.

NPL 1 discloses the influence of the stride length on the plantar pressure and the joint moment. NPL 2 discloses independent effects of gait speed and stride length on stability and falling risk. By using the methods of NPL 1 and NPL 2, it is possible to evaluate the exercise function such as the knee joint load and the gait stability by verifying the measurement data such as the stride length and the gait speed.

In a facility such as a hospital or a sports gym, since the facility is placed in an environment different from daily life, the movement such as walking may exhibit a different aspect from daily life. In such a case, an evaluation result that does not reflect the original exercise function may be obtained.

PTL 1 discloses a biometric data management system that calibrates biometric data measured at a hospital or home with each other. The system of PTL 1 includes a first biometric device and a second biometric device that measure biometric data of a subject. The first biometric device is used in a hospital. The second biometric device is used in an environment such as a home different from that of the first biometric device. The system of PTL 1 stores an error between the biometric data of the subject measured by the first biometric device and the biometric data of the subject measured by the second biometric device in advance as a calibration value. The system of PTL 1 calibrates biometric data of a subject with the calibration value.

### CITATION LIST

### Patent Literature

PTL 1: JP 2014-180361 A

### Non Patent Literature

NPL 1: Lara Allet, et al., “The influence of stride-length on plantar foot-pressures and joint moments”, Gait & Posture 34, (2011), pp. 300-306.
NPL 2: D. D. Espy, et al., “Independent Influence of Gait Speed and Step Length on Stability and Fall Risk”, Gait Posture, 2010 July, 32(3), 378-82.

### SUMMARY OF INVENTION

### Technical Problem

In the method of PTL 1, biometric data measured in different environments such as a hospital and a home is calibrated with a previously stored calibration value. In the method of PTL 1, since the biometric data is calibrated with a constant calibration value, the biometric data cannot be accurately calibrated unless the difference between the environments is clarified in advance. In daily life such as at home, in order to make the measurement environment of the biometric data constant, it is necessary to keep the body stationary. When evaluating an exercise function such as walking, the body cannot be kept stationary.

An object of the present disclosure is to provide a biometric information processing device and the like capable of correcting a difference in biometric data for evaluating an exercise function measured in different environments.

### Solution to Problem

A biometric information processing device according to an aspect of the present disclosure includes an extraction unit that extracts a daily measurement value of a common measurement item common to facility measurement data measured in a facility from daily measurement data measured using the sensor data regarding movement of a foot of a user, and a correction unit that corrects a facility measurement value of the common measurement item stored in advance based on the extracted daily measurement value of the common measurement item.

In a biometric information processing method according to an aspect of the present disclosure, a computer extracts a daily measurement value of a common measurement item common to facility measurement data measured in a facility from daily measurement data measured using sensor data related to a motion of a foot of a user, and corrects a facility measurement value of the common measurement item stored in advance based on the extracted daily measurement value of the common measurement item.

A program according to an aspect of the present disclosure causes a computer to execute extracting a daily measurement value of a common measurement item common to facility measurement data measured in a facility from daily measurement data measured using the sensor data regarding movement of a foot of a user, and correcting a facility measurement value of the common measurement item stored in advance based on the extracted daily measurement value of the common measurement item.

### Advantageous Effects of Invention

According to the present disclosure, it is possible to provide a biometric information processing device or the like capable of correcting a difference in biometric data for evaluating an exercise function measured in different environments.

### BRIEF DESCRIPTION OF DRAWINGS

FIG. 1 is a block diagram illustrating an example of a configuration of an information processing system according to a first example embodiment.

FIG. 2 is a conceptual diagram illustrating an arrangement example of measurement devices of the information processing system according to the first example embodiment.

FIG. 3 is a conceptual diagram for explaining a coordinate system set in the measurement device of the information processing system according to the first example embodiment.

FIG. 4 is a conceptual diagram for explaining a human body surface used in the description of the information processing system according to the first example embodiment.

FIG. 5 is a conceptual diagram for explaining a gait cycle used in the description of the information processing system according to the first example embodiment.

FIG. 6 is a conceptual diagram for explaining an example of daily measurement items of the information processing system according to the first example embodiment.

FIG. 7 is a conceptual diagram for explaining another example of daily measurement items of the information processing system according to the first example embodiment.

FIG. 8 is a conceptual diagram for explaining still another example of daily measurement items of the information processing system according to the first example embodiment.

FIG. 9 is a block diagram illustrating an example of a configuration of the measurement device of the information processing system according to the first example embodiment.

FIG. 10 is a block diagram illustrating an example of a configuration of a biometric information processing device of the information processing system according to the first example embodiment.

FIG. 11 is a diagram illustrating an example of daily measurement data measured by the biometric information processing device of the information processing system according to the first example embodiment.

FIG. 12 is an example of facility measurement values of common measurement items stored in the biometric information processing device of the information processing system according to the first example embodiment.

FIG. 13 is an example of a frequency distribution of daily measurement values of stride lengths measured by the biometric information processing device of the information processing system according to the first example embodiment.

FIG. 14 is an example of a frequency distribution of daily measurement values of a gait speed measured by the biometric information processing device of the information processing system according to the first example embodiment.

FIG. 15 is an example of correction values of measurement values of common measurement items calculated by the biometric information processing device of the information processing system according to the first example embodiment.

FIG. 16 is a flowchart for explaining an example of the operation of the biometric information processing device of the information processing system according to the first example embodiment.

FIG. 17 is a block diagram illustrating an example of a configuration of an information processing system according to a second example embodiment.

FIG. 18 is a block diagram illustrating an example of a configuration of a biometric information processing device of the information processing system according to the second example embodiment.

FIG. 19 is an example of facility measurement values of a common measurement item and a facility measurement item stored in the biometric information processing device of the information processing system according to the second example embodiment.

FIG. 20 is an example of correction values of facility measurement values of facility measurement items calculated by the biometric information processing device of the information processing system according to the first example embodiment.

FIG. 21 is a flowchart for explaining an example of the operation of the biometric information processing device of the information processing system according to the second example embodiment.

FIG. 22 is a conceptual diagram for explaining Application Example 1 of the information processing system according to the second example embodiment.

FIG. 23 is a conceptual diagram for explaining Application Example 1 of the information processing system according to the second example embodiment.

FIG. 24 is a conceptual diagram for explaining Application Example 2 of the information processing system according to the second example embodiment.

FIG. 25 is a block diagram illustrating an example of a configuration of a biometric information processing device according to a third example embodiment.

FIG. 26 is a block diagram illustrating an example of a hardware configuration that executes control and processing according to each example embodiment.

### EXAMPLE EMBODIMENT

Hereinafter, example embodiments of the present invention will be described with reference to the drawings. However, the example embodiments described below may be technically limited for carrying out the present invention, but the scope of the invention is not limited to the following. In all the drawings used in the following description of the example embodiment, the same reference numerals are given to the same parts unless there is a particular reason. In the following example embodiments, repeated description of similar configurations and operations may be omitted. The directions of the arrows in the drawings illustrate an example, and do not limit the directions of signals, and the like between blocks.

### First Example Embodiment

First, an information processing system according to a first example embodiment will be described with reference to the drawings. The information processing system of the present example embodiment measures measurement data on a daily basis (also referred to as daily measurement data) using sensor data acquired by a sensor installed on a foot portion of the user. The information processing system of the present example embodiment extracts a measurement item (also referred to as a common measurement item) common to measurement data (also referred to as facility measurement data) measured in a facility such as a sports gym or a hospital from the measured daily measurement data. The daily measurement data is shallow data having a large data amount but a limited information amount. The facility measurement data is deep data having a small data amount but a large information amount. The information processing system of the present example embodiment corrects the facility measurement data based on the daily measurement data having a large data amount.

(Configuration)

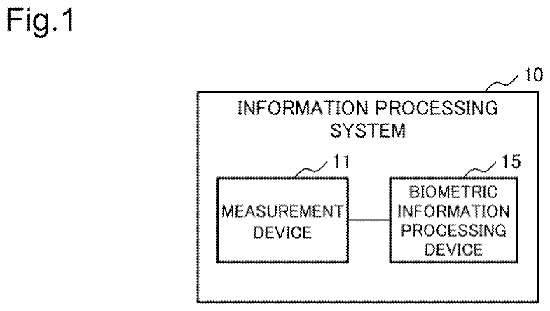

FIG. 1 is a block diagram illustrating an example of a configuration of an information processing system 10 according to the present example embodiment. The information processing system 10 includes a measurement device 11 and a biometric information processing device 15. The measurement device 11 and the biometric information processing device 15 may be connected by wire or wirelessly. The measurement device 11 and the biometric information processing device 15 may be configured by a single device. The information processing system 10 may include only the biometric information processing device 15 except for the measurement device 11 from the configuration of the information processing system 10.

The measurement device 11 is installed on the foot portion. The measurement device 11 measures acceleration (also referred to as spatial acceleration) and angular speed (also referred to as spatial angular speed) as physical quantities related to the movement of the foot of the user wearing footwear such as shoes. The physical quantity related to the movement of the foot measured by the measurement device 11 includes a speed, an angle, and a position (trajectory) calculated by integrating the acceleration and the angular speed, in addition to the acceleration and the angular speed. The measurement device 11 converts the measured physical quantity into digital data (also referred to as sensor data). The measurement device 11 transmits the converted sensor data to the biometric information processing device 15. Sensor data such as acceleration and angular speed generated by the measurement device 11 is also referred to as a gait parameter. For example, a speed, an angle, a trajectory, and the like calculated by integrating acceleration and angular speed are also included in the gait parameter. For example, an angle of the sole with respect to the ground (also referred to as a plantar angle) is also included in the gait parameter.

The measurement device 11 is achieved by, for example, an inertial measurement device including an acceleration sensor and an angular speed sensor. An example of the inertial measurement device is an inertial measurement unit (IMU). The IMU includes a three-axis acceleration sensor and a three-axis angular speed sensor. The measurement device 11 may include a sensor other than the acceleration sensor and the angular speed sensor. Another example of the inertial measurement device includes a vertical gyro (VG), an attitude heading (AHRS), and a global positioning system (Global Positioning System/Inertial Navigation System).

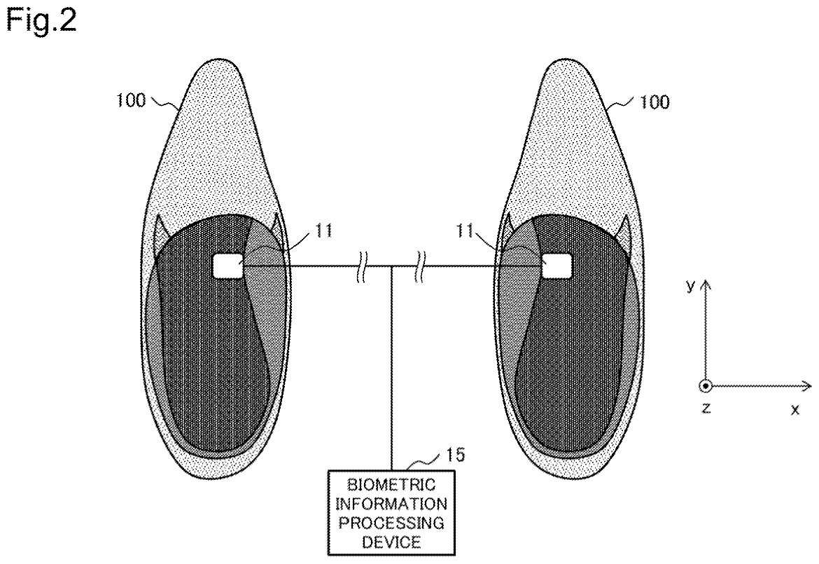

FIG. 2 is a conceptual diagram illustrating an example of installing the measurement device 11 in a shoe 100. In the example of FIG. 2, the measurement device 11 is installed at a position relevant to the back side of the arch of foot. For example, the measurement device 11 is installed in an insole inserted into the shoe 100. For example, the measurement device 11 is installed on the bottom surface of the shoe 100. For example, the measurement device 11 is embedded in the main body of the shoe 100. The measurement device 11 may be detachable from the shoe 100 or may not be detachable from the shoe 100. The measurement device 11 may be installed at a position other than the back side of the arch of the foot as long as the sensor data regarding the movement of the foot can be acquired. The measurement device 11 may be installed on a sock worn by the user or a decorative article such as an anklet worn by the user. The measurement device 11 may be directly attached to the foot or may be embedded in the foot. FIG. 2 illustrates an example in which the measurement devices 11 are installed on the shoes 100 of both feet. The measurement device 11 may be installed on at least one foot portion. If the measurement devices 11 are installed on the shoes 100 of both feet, evaluation can be performed based on sensor data measured by the measurement devices 11 installed on the left and right feet.

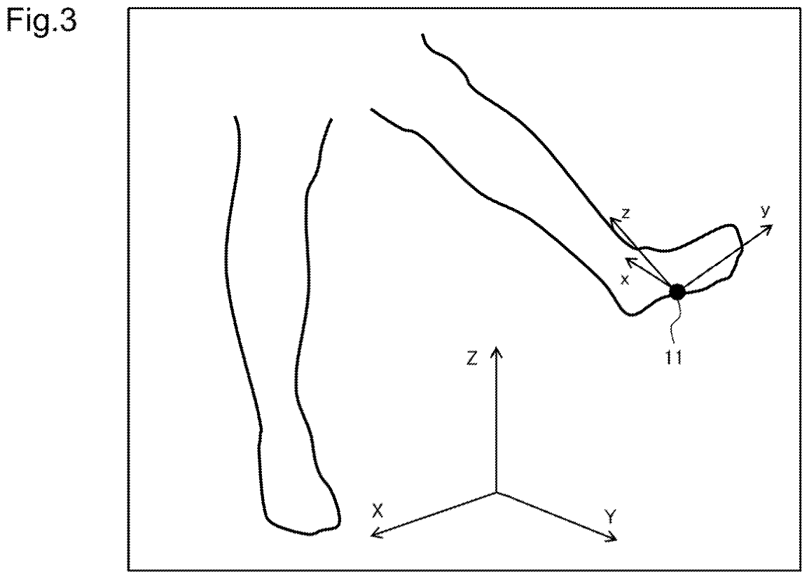

FIG. 3 is a conceptual diagram for explaining a local coordinate system (x axis, y axis, z axis) set in the measurement device 11 and a world coordinate system (X axis, Y axis, Z axis) set with respect to the ground. In the world coordinate system (X axis, Y axis, Z axis), in a state where the user is standing upright, a lateral direction of the user is set to an X-axis direction (rightward direction is positive), a front direction of the user (traveling direction) is set to a Y-axis direction (forward direction is positive), and a gravity direction is set to a Z-axis direction (vertically upward direction is positive). In the present example embodiment, the local coordinate system including an x direction, a y direction, and a z direction based on the measurement device 11 is set.

The biometric information processing device 15 receives sensor data from the measurement device 11. The biometric information processing device 15 measures daily measurement data using the received sensor data. For example, the daily measurement data includes a stride length, a gait speed, a maximum dorsiflexion angle, a maximum plantarflexion angle, a foot elevation height, a diversion amount, and a foot angle.

The biometric information processing device 15 extracts a value (also referred to as a daily measurement value) of the daily measurement data of the common measurement item common to the facility measurement data among the measured daily measurement data. The common measurement item is set in advance according to the exercise function to be evaluated. For example, the stride length and the gait speed are set as the common measurement items. For example, the biometric information processing device 15 extracts daily measurement values of the stride length and the gait speed which are common measurement items. Daily measurement data other than the stride length and the gait speed may be set as the common measurement item.

The biometric information processing device 15 calculates a correction value of the extracted common measurement item. For example, the biometric information processing device 15 calculates, as the correction value, a difference between the daily measurement value of the common measurement item extracted from the daily measurement data measured based on the sensor data and the value (also referred to as a facility measurement value) of the facility measurement data of the common measurement item stored in advance. For example, the biometric information processing device 15 calculates, as the correction value, a deviation between the representative value in the distribution of the daily measurement values of the common measurement items extracted from the daily measurement data and the facility measurement value of the common measurement item stored in advance.

The biometric information processing device 15 outputs the correction value of the common measurement item. For example, the biometric information processing device 15 outputs a correction value of the facility measurement value of the common measurement item. For example, the biometric information processing device 15 outputs the correction value to a terminal device (not illustrated) browsable in the facility in which the facility measurement data is measured. For example, the correction value output to the terminal device is displayed on the screen of the terminal device. For example, a person who has confirmed the correction value displayed on the screen of the terminal device can recognize a difference between the facility measurement data and the daily measurement data. For example, the biometric information processing device 15 may be configured to output the correction value of the common measurement item to a display device (not illustrated) or an external system.

Next, matters related to gait verified in the present example embodiment will be described with reference to the drawings. The following matters may be different from the general definition regarding gait verified in the present example embodiment. The following matters also apply to other example embodiments of the present disclosure.

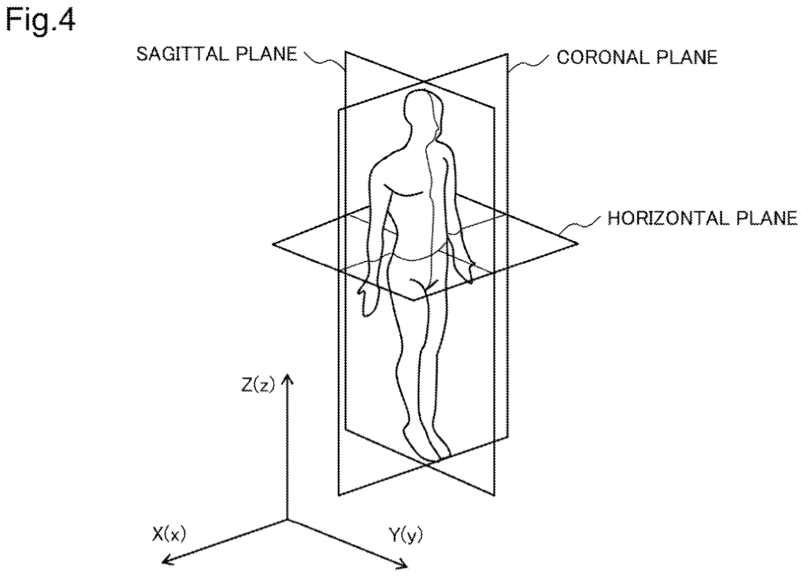

FIG. 4 is a conceptual diagram for explaining a surface (also referred to as a human body surface) set for the human body. In the present example embodiment, a sagittal plane dividing the body into left and right, a coronal plane dividing the body into front and rear, and a horizontal plane dividing the body horizontally are defined. In the upright state as illustrated in FIG. 4, the world coordinate system coincides with the local coordinate system. In the present example embodiment, rotation in the sagittal plane with the x axis as the rotation axis is defined as roll, rotation in the coronal plane with the y axis as the rotation axis is defined as pitch, and rotation in the horizontal plane with the z axis as the rotation axis is defined as yaw. A rotation angle in a sagittal plane with the x axis as a rotation axis is defined as a roll angle, a rotation angle in a coronal plane with the y axis as a rotation axis is defined as a pitch angle, and a rotation angle in a horizontal plane with the z axis as a rotation axis is defined as a yaw angle. In the present example embodiment, when the body is viewed from the right side surface, clockwise rotation in the sagittal plane is defined as positive, and counterclockwise rotation in the sagittal plane is defined as negative.

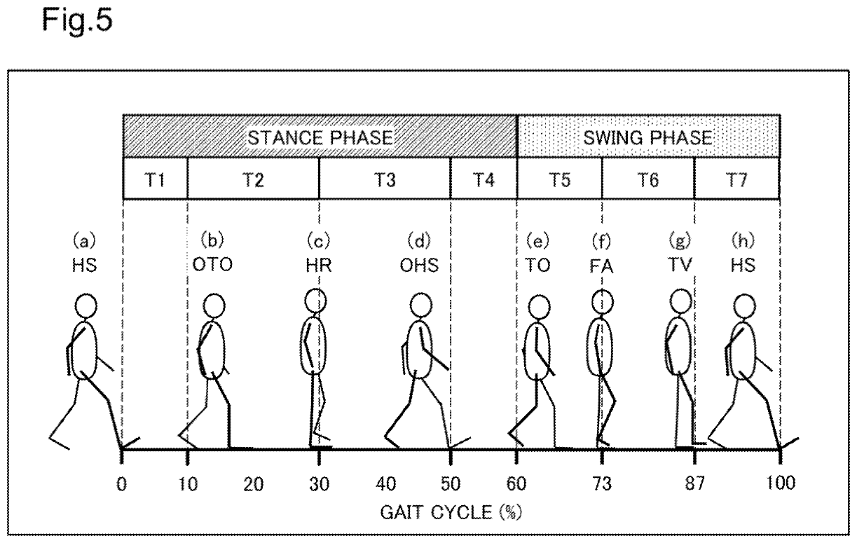

FIG. 5 is a conceptual diagram for explaining one gait cycle with the right foot as a reference. One gait cycle based on the left foot is also similar to that of the right foot. The horizontal axis of FIG. 5 is a gait cycle normalized with one gait cycle of the right foot as 100%, with a time point at which the heel of the right foot lands on the ground as a starting point and a time point at which the heel of the right foot next lands on the ground as an ending point. The one gait cycle of one foot is roughly divided into a stance phase in which at least a part of the back side of the foot is in contact with the ground and a swing phase in which the back side of the foot is separated from the ground. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. FIG. 5 is an example, and does not limit the periods constituting one gait cycle, the names of these periods, and the like.

As illustrated in FIG. 5, in gait, a plurality of events (also referred to as gait events) occur. (a) of FIG. 5 represents an event in which the heel of the right foot touches the ground (HS: Heel Strike). (b) of FIG. 5 illustrates an event (opposite toe off) in which the toe of the left foot is separated from the ground while the sole of the right foot is in contact with the ground (OTO: Opposite Toe Off). (c) of FIG. 5 illustrates an event (heel rise) in which the heel of the right foot is lifted while the sole of the right foot is in contact with the ground (HR: Heel Rise). (d) of FIG. 5 is an event in which the heel of the left foot is grounded (OHS: Opposite Heel Strike). (e) of FIG. 5 illustrates an event (toe off) in which the toe of the right foot is separated from the ground in a state where the sole of the left foot is in contact with the ground (TO: Toe Off). (f) of FIG. 5 illustrates an event (foot adjacent) in which the left foot and the right foot cross each other in a state where the sole of the left foot is grounded (FA: Foot Adjacent). (g) of FIG. 5 illustrates an event (tibia vertical) in which the tibia of the right foot is substantially perpendicular to the ground with the sole of the left foot in contact with the ground (TV: Tibia Vertical). (h) of FIG. 5 illustrates an event in which the heel of the right foot touches the ground (HS: Heel Strike). (h) of FIG. 5 corresponds to the ending point of the gait cycle starting from (a) of FIG. 5 and corresponds to the starting point of the next gait cycle. FIG. 5 is an example, and does not limit events that occur accompanying gait or names of these events.

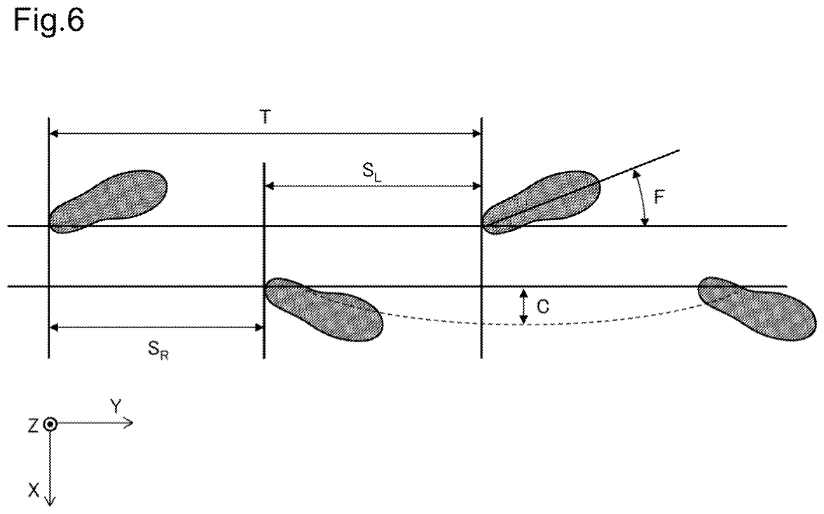

FIG. 6 is a conceptual diagram for explaining a gait parameter calculated by the biometric information processing device 15. FIG. 6 illustrates a right-foot step length S_{R}, a left-foot step length SL, a stride length T, a diversion amount C, and a foot angle F. The right-foot step length S_{R} is a difference in the Y-coordinate between the heel of the right foot and the heel of the left foot when the state in which the sole of the left foot is in contact with the ground transitions to the state in which the heel of the right foot swung out in the traveling direction lands. The left-foot step length SL is a difference between the Y-coordinates of the heel of the left foot and the heel of the right foot when the state in which the sole of the right foot is in contact with the ground transitions to the state in which the heel of the left foot swung out in the traveling direction lands. The stride length T is the sum of the right-foot step length S_{R} and the left-foot step length SL. The diversion amount C is the degree of diversion of the foot in the horizontal plane (XY plane). Here, a straight line connecting the position of the measurement device 11 in a state where the sole of one foot is in contact with the ground and the position of the measurement device 11 when the sole of the foot swung out in the traveling direction transitions to a state where the sole of the foot is in contact with the ground is defined as a traveling axis. The diversion amount C is a distance between the measurement device 11 and the traveling axis at a timing when the foot is farthest from the traveling axis in the horizontal plane in a period from a state in which the sole of one foot is in contact with the ground to a state in which the sole of the foot swung out in the traveling direction is in contact with the ground again. The foot angle F is an angle formed by the center line of the foot and the traveling axis (Y axis) in a state where the sole surface is in contact with the ground.

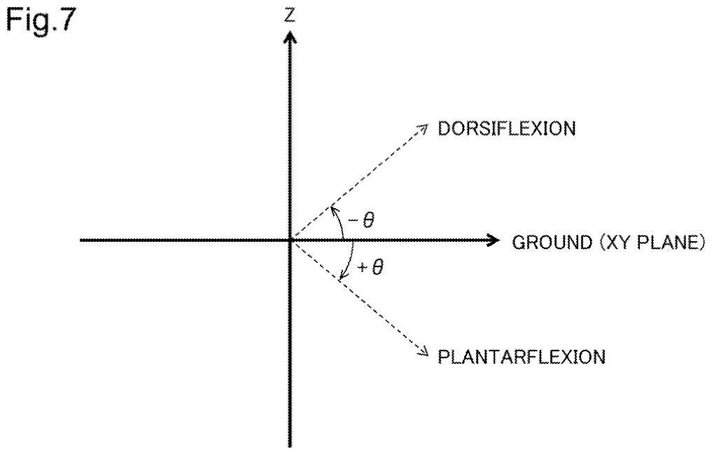

FIG. 7 is a conceptual diagram for explaining the plantar angle. The plantar angle is the angle of the plantar surface relative to the ground (XY plane). Regarding the plantar angle, a state in which the toe is directed upward (dorsiflexion) is defined as minus, and a state in which the toe is directed downward (plantarflexion) is defined as plus. In one gait cycle, the angle at which the absolute value of the plantar angle in the dorsiflexion state becomes maximum is referred to as a maximum dorsiflexion angle. In one gait cycle, the angle at which the absolute value of the plantar angle in the plantarflexion state becomes maximum is referred to as a maximum plantarflexion angle.

For example, the biometric information processing device 15 calculates the plantar angle using the acceleration in the X-axis direction and the acceleration in the Y-axis direction. For example, the biometric information processing device 15 calculates the plantar angle around each of the X axis, the Y axis, and the Z axis by integrating the values of the angular speed having each of these axes as the central axis. The acceleration data and the angular speed data include high-frequency and low-frequency noises that change in various directions. Therefore, a low-pass filter and a high-pass filter are applied to the acceleration data and the angular speed data to remove a high-frequency component and a low-frequency component. If the high-frequency component and the low-frequency component are removed, the accuracy of the sensor data on which noise easily rides can be improved. It is also possible to improve the accuracy of the sensor data by applying a complementary filter to each of the acceleration data and the angular speed data and taking a weighted average. The measurement device 11 may be configured to measure the plantar angle.

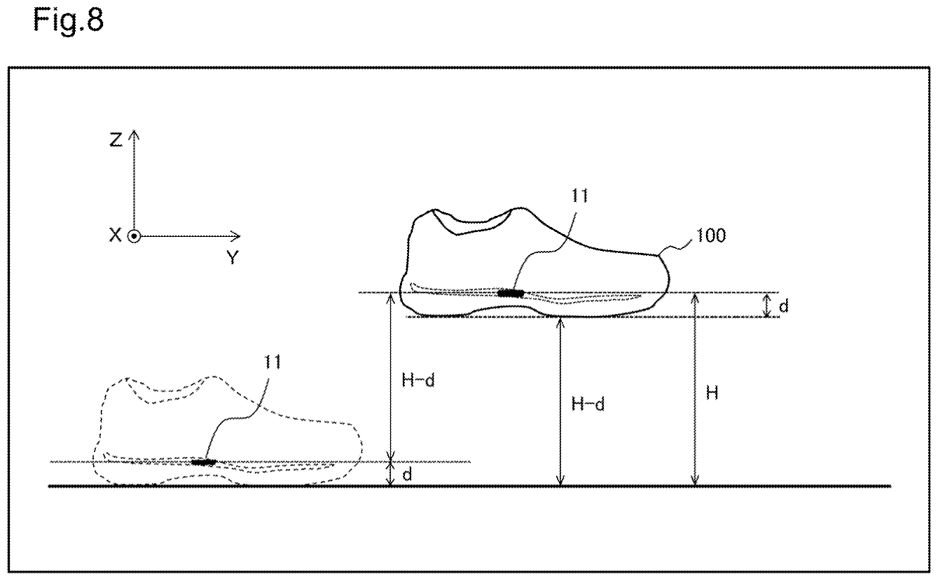

FIG. 8 is a conceptual diagram for explaining the foot elevation height. The foot elevation height is a height of the sole with respect to the ground. FIG. 8 illustrates a state in which, in one gait cycle, a transition is made from a position (broken line) where the sole is in contact with the ground to a position (solid line) where the foot elevation height with respect to the ground of the sole is maximum. A foot elevation height H corresponds to the position of the measurement device 11 in the Z direction. That is, the foot elevation height H substantially coincides with the height in the Z direction of the sole of the user walking while wearing the shoes 100. In the example of FIG. 8, the foot elevation height H can be calculated by second-order integration of acceleration in the Z direction. The measurement device 11 is installed at a position of an initial height d in the shoe 100. Therefore, when a state in which the sole is in contact with the ground is used as a reference, the height of the sole in the Z direction changes by H−d. The height of the sole of the shoe 100 also corresponds to H−d. In the evaluation of the exercise function based on the foot elevation height, it may be preferable to set H−d as the foot elevation height.

(Data Acquisition Device)

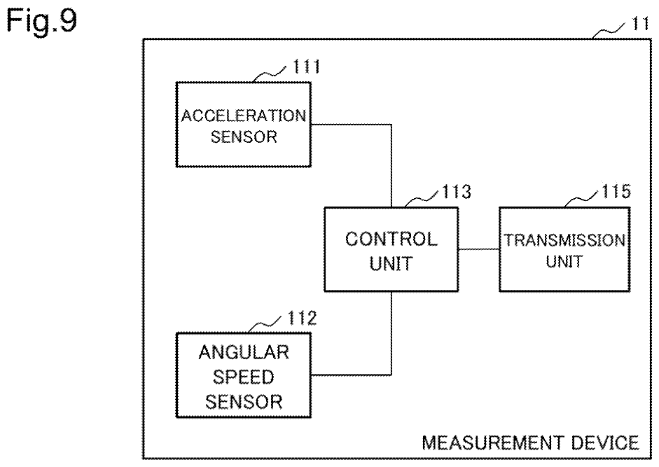

Next, details of the measurement device 11 will be described with reference to the drawings. FIG. 9 is a block diagram illustrating an example of a detailed configuration of the measurement device 11. The measurement device 11 includes an acceleration sensor 111, an angular speed sensor 112, a control unit 113, and a transmission unit 115. The measurement device 11 includes a power supply (not illustrated).

The acceleration sensor 111 is a sensor that measures accelerations (also referred to as spatial accelerations) in three axial directions. The acceleration sensor 111 outputs the measured acceleration to the control unit 113. For example, a sensor of a piezoelectric type, a piezoresistive type, a capacitance type, or the like can be used as the acceleration sensor 111. The sensor used for the acceleration sensor 111 is not limited to the measurement method as long as the sensor can measure acceleration.

The angular speed sensor 112 is a sensor that measures an angular speed (also referred to as a spatial angular speed) around three axes. The angular speed sensor 112 outputs the measured angular speed to the control unit 113. For example, a sensor of a vibration type, a capacitance type, or the like can be used as the angular speed sensor 112. The sensor used for the angular speed sensor 112 is not limited to the measurement method as long as the sensor can measure the angular speed.

The control unit 113 acquires accelerations in three axial directions from the acceleration sensor 111. The control unit 113 acquires angular speeds around three axes from the angular speed sensor 112. The control unit 113 converts the acquired acceleration and angular speed into digital data. The control unit 113 outputs the converted digital data (also referred to as sensor data) to the transmission unit 115. The sensor data includes at least acceleration data converted from analog data to digital data and angular speed data converted from analog data to digital data. The acceleration data converted into the digital data includes acceleration vectors in three axial directions. The angular speed data converted into the digital data includes angular speed vectors in three axial directions. The acceleration data and the angular speed data are associated with acquisition times of the data. The control unit 113 may be configured to output sensor data obtained by adding correction such as a mounting error, temperature correction, and linearity correction to the acquired acceleration data and angular speed data. The control unit 113 may be configured to measure angle data and a plantar angle around three axes using the acquired acceleration data and angular speed data.

For example, the control unit 113 is a microcomputer or a microcontroller that controls or processes the measurement device 11. For example, the control unit 113 includes a central processing unit (CPU), a random access memory (RAM), a read only memory (ROM), a flash memory, and the like. The control unit 113 controls the acceleration sensor 111 and the angular speed sensor 112 to measure the angular speed and the acceleration. For example, the control unit 113 performs analog-to-digital conversion (AD conversion) on physical quantities (analog data) such as the measured angular speed and acceleration, and stores the converted digital data in the flash memory. The physical quantity (analog data) measured by the acceleration sensor 111 and the angular speed sensor 112 may be converted into digital data in each of the acceleration sensor 111 and the angular speed sensor 112. The digital data stored in the flash memory is output to the transmission unit 115 at a predetermined timing.

The transmission unit 115 acquires sensor data from the control unit 113. The transmission unit 115 transmits the acquired sensor data to the biometric information processing device 15. The transmission unit 115 may transmit the sensor data to the biometric information processing device 15 via a wire such as a cable, or may transmit the sensor data to the biometric information processing device 15 via wireless communication. For example, the transmission unit 115 is configured to transmit the sensor data to the biometric information processing device 15 via a wireless communication function (not illustrated) conforming to a standard such as Bluetooth (registered trademark) or WiFi (registered trademark). The communication function of the transmission unit 115 may conform to a standard other than Bluetooth (registered trademark) or WiFi (registered trademark).

(Biometric Information Processing Device)

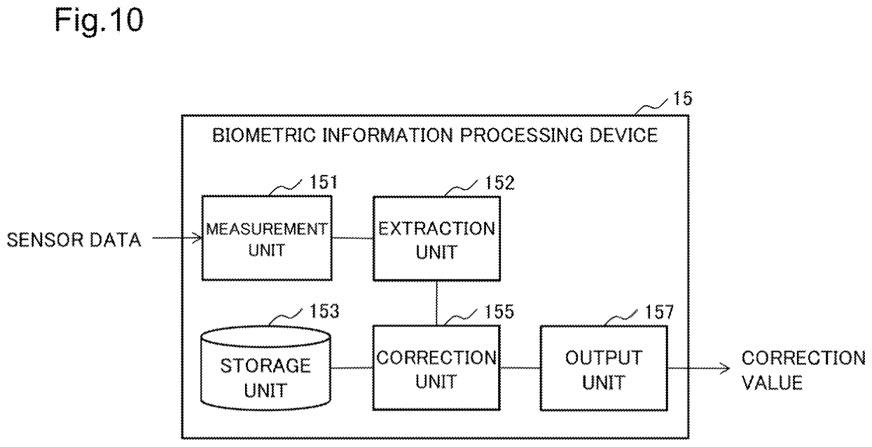

Next, details of the biometric information processing device 15 included in the information processing system 10 will be described with reference to the drawings. FIG. 10 is a block diagram illustrating an example of a configuration of the biometric information processing device 15. The biometric information processing device 15 includes a measurement unit 151, an extraction unit 152, a storage unit 153, a correction unit 155, and an output unit 157.

The measurement unit 151 acquires sensor data from the measurement device 11 installed on the footwear worn by the pedestrian. The measurement unit 151 measures daily measurement data using the acquired sensor data. For example, the measurement unit 151 measures daily measurement data such as a stride length, a gait speed, a maximum dorsiflexion angle, a maximum plantarflexion angle, a foot elevation height, a diversion amount, and a foot angle. Hereinafter, an example of a method of measuring daily measurement data by the measurement unit 151 will be described.

First, the measurement unit 151 converts the coordinate system of the acquired sensor data from the local coordinate system to the world coordinate system. When the user is standing upright, the local coordinate system (x axis, y axis, z axis) and the world coordinate system (X axis, Y axis, Z axis) coincide. On the other hand, since the spatial posture of the measurement device 11 changes while the user is walking, the local coordinate system (x axis, y axis, z axis) and the world coordinate system (X axis, Y axis, Z axis) do not match. Therefore, the measurement unit 151 converts the sensor data acquired by the measurement device 11 from the local coordinate system (x axis, y axis, z axis) of the measurement device 11 into the world coordinate system (X axis, Y axis, Z axis).

The measurement unit 151 generates time-series data associated with gait of the user wearing the footwear on which the measurement device 11 is installed, using the sensor data converted into the world coordinate system. The measurement unit 151 extracts gait waveform data for one gait cycle from the generated time-series data. For example, the measurement unit 151 generates time-series data such as a spatial acceleration and a spatial angular speed. The measurement unit 151 integrates the spatial acceleration and the spatial angular speed to generate time-series data such as a spatial speed, a spatial angle, a plantar angle, and a spatial trajectory. The timing at which the measurement unit 151 generates the time-series data can be arbitrarily set. For example, the measurement unit 151 generates time-series data at a predetermined timing set in accordance with a general gait cycle or a gait cycle unique to the user. For example, the measurement unit 151 generates time-series data at predetermined time intervals set in accordance with the gait cycle. For example, the measurement unit 151 continues to generate time-series data during a period in which the user keeps walking. For example, the measurement unit 151 may generate time-series data at a specific time.

The measurement unit 151 detects a gait event of the user walking while wearing the footwear on which the measurement device 11 is installed from the generated gait waveform data. For example, the measurement unit 151 extracts a feature for each gait event from a gait waveform of a physical quantity related to the movement of the foot. For example, the measurement unit 151 detects the timing of the extracted feature for each gait event as the timing of each gait event.

For example, the measurement unit 151 measures, as the stride length, a movement distance of the measurement device 11 in the Y direction at a timing of a continuous heel strike, a continuous toe off, or the like. For example, the measurement unit 151 measures the gait speed by integrating the acceleration in the Y direction. For example, the measurement unit 151 measures, as the maximum dorsiflexion angle, an angle at which the absolute value of the plantar angle is the maximum in the dorsiflexion direction. The measurement unit 151 measures, as the maximum plantarflexion angle, an angle at which the absolute value of the plantar angle is maximum in the plantarflexion direction. For example, the measurement unit 151 calculates the foot elevation height by performing second-order integration on the Z-direction acceleration. For example, the measurement unit 151 obtains a trajectory of a position in the X direction by performing second-order integration of acceleration in the X direction for one gait cycle. The measurement unit 151 measures, as a diversion amount, a distance at which the distance between the position in the X direction and the traveling axis is maximized. For example, the measurement unit 151 measures, as the foot angle, an angle formed by the center line of the foot and the traveling axis (Y axis) in a state where the sole is grounded. The measurement method of the daily measurement data described here is an example, and the measurement method of the daily measurement data by the measurement unit 151 is not limited.

For example, the measurement unit 151 may detect the toe off, the heel strike, and the foot adjacent as gait events, and measure the stride length based on these gait events. The foot adjacent corresponds to timing at which the toe of one foot passes through the position of the midpoint between the toe and the heel of the other foot. The measurement unit 151 extracts a section between the toe off and the heel strike from the gait waveform of the Y-direction trajectory for one gait cycle as the gait waveform of the Y-direction trajectory for one step. The measurement unit 151 calculates the absolute value of the difference between the spatial position at the foot adjacent and the spatial position at the toe off using the gait waveform of the Y-direction trajectory of one step. The absolute value of the difference between the spatial position at the foot adjacent and the spatial position at the toe off corresponds to the left-foot step length SL (also referred to as a first step length) in a state where the left foot is in the front and the right foot is in the back. The measurement unit 151 calculates the absolute value of the difference between the spatial position at the timing of the foot adjacent and the spatial position at the heel strike by using the gait waveform of the Y-direction trajectory of one step. The absolute value of the difference between the spatial position at the timing of the foot adjacent and the spatial position at the heel strike corresponds to the right-foot step length S_{R} (also referred to as a second step length) in a state where the right foot is in front and the left foot is in back. The sum of the right-foot step length S_{R} and the left-foot step length SL corresponds to the stride length. According to this method, the step length of each foot can be individually measured.

The extraction unit 152 extracts a daily measurement value of a common measurement item with the facility measurement data from the daily measurement data measured by the measurement unit 151. For example, the extraction unit 152 extracts daily measurement values of the stride length and the gait speed as common measurement items.

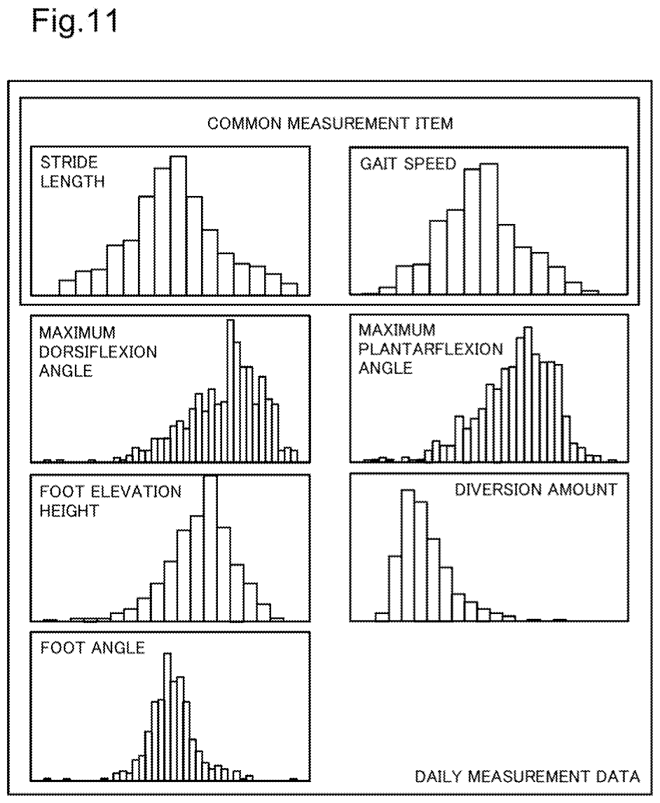

For example, the extraction unit 152 extracts a representative value of the daily measurement value of the common measurement item with the facility measurement data from the daily measurement data continuously measured by the measurement unit 151. FIG. 11 is a diagram summarizing frequency distributions of daily measurement values measured over a plurality of dates and times. FIG. 11 includes frequency distributions of daily measurement values of stride length, gait speed, maximum dorsiflexion angle, maximum plantarflexion angle, foot elevation height, diversion amount, and foot angle. In FIG. 11, the scales on the horizontal axis and the vertical axis are omitted. In the example of FIG. 11, the stride length and the gait speed are common measurement items. For example, the extraction unit 152 extracts a representative value of the daily measurement values of the stride length and the gait speed as the common measurement item.

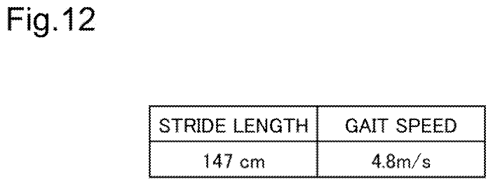

The storage unit 153 stores the facility measurement value of the common measurement item. For example, the storage unit 153 stores facility measurement values of the stride length and the gait speed. FIG. 12 is an example of the facility measurement value stored in the storage unit 153. In the example of FIG. 12, a facility measurement value 147 cm (centimeter) of the stride length and a facility measurement value 4.8 m/s (meter per second) of the gait speed are stored in the storage unit 153. The facility measurement value of the common measurement item stored in the storage unit 153 can be updated at an arbitrary timing. For example, the facility measurement value of the common measurement item stored in the storage unit 153 is updated according to data transmitted from a facility such as a sports gym or a hospital. A method for updating the facility measurement value of the common measurement item stored in the storage unit 153 is not particularly limited.

The correction unit 155 calculates the daily measurement value of the common measurement item and the correction value of the facility measurement value. For example, the correction unit 155 calculates a difference between the daily measurement value and the facility measurement value of the common measurement item as the correction value. For example, the correction unit 155 calculates a deviation between the representative value of the distribution of the daily measurement values of the common measurement item and the facility measurement value as the correction value. For example, as the representative value of the distribution of the daily measurement values of the common measurement item, the correction unit 155 uses, as the representative value, an average value such as an arithmetic mean, a geometric mean, or a harmonic mean of a plurality of daily measurement values constituting the distribution. For example, the correction unit 155 uses, as a representative value of the distribution of the daily measurement values of the common measurement item, a mode value or a median value of a plurality of daily measurement values constituting the distribution.

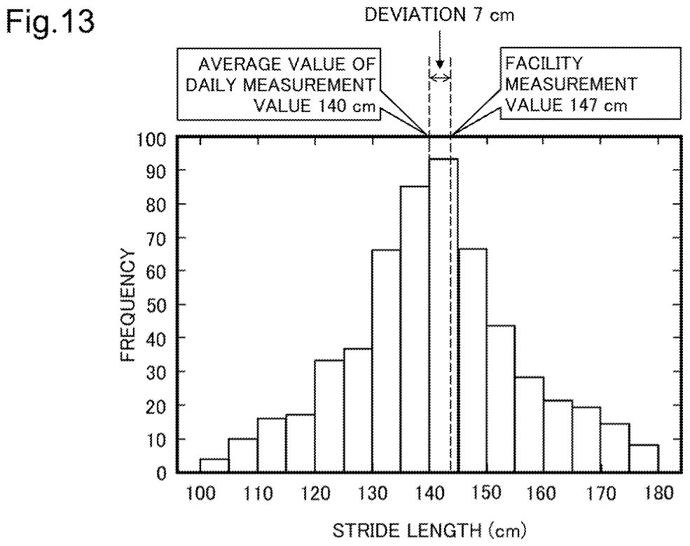

FIG. 13 is an example of a frequency distribution of the daily measurement value of the stride length. The daily measurement value of the stride length in FIG. 13 is a distribution of 561 samples measured in walking for about 40 days and from which outliers have been removed. In the example of FIG. 13, the average value of the daily measurement values of the stride length is 140 cm. On the other hand, the facility average value of the stride is 147 cm. The deviation between the average value of the distribution of the daily measurement values of the stride length and the facility measurement value is 7 cm.

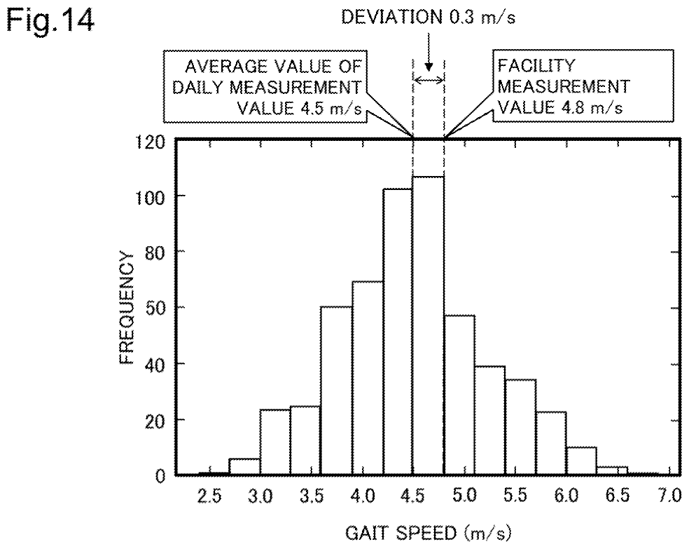

FIG. 14 is an example of frequency distribution of daily measurement values of a gait speed. The daily measurement value of the gait speed in FIG. 13 is a value measured at the same timing as in FIG. 14. In the example of FIG. 14, the average value of the daily measurement values of the gait speed is 4.8 m/s. On the other hand, the facility average value of the gait speed is 4.5 m/s. The deviation between the average value of the distribution of the daily measurement values of the gait speed and the facility measurement value is 0.3 m/s.

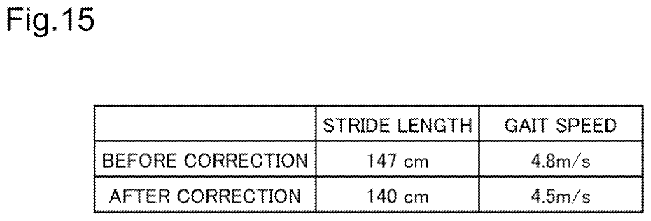

FIG. 15 is a table summarizing values of common measurement items before and after correction by the correction unit 155. In the table of FIG. 15, a row of facility measurement values of the stride length and the gait speed before correction and a row of correction values of the stride length and the gait speed after correction are vertically arranged. For example, a trainer who instructs the exercise to the user who is the acquisition source of the data of FIG. 15 can review the training menu created based on the data before correction by comparing the stride length and the gait speed before and after correction.

The facility measurement value is measured under observation in the facility, and thus tends to be different from the daily measurement value. For example, when the target person tries to walk in a better posture than usual, the stride tends to increase and the gait speed tends to increase. In the examples of FIGS. 13 to 15, it is presumed that such a tendency appears. On the other hand, for example, when the target person walks to show a feeling of being disordered more than usual, the stride tends to be small and the gait speed tends to be slow. In the present example embodiment, the facility measurement value can be corrected using the daily measurement value based on natural gait. Therefore, according to the present example embodiment, the original exercise function in the daily environment can be reflected in the detailed evaluation of the exercise function in the facility.

The output unit 157 outputs the correction value of the common measurement item. For example, the output unit 157 outputs the value of the common measurement item corrected by the correction value. For example, the output unit 157 displays the values of the common measurement item and the facility measurement item corrected by the correction value on the screen of the mobile terminal carried by the user. For example, the output unit 157 outputs the correction value to a terminal device browsable in the facility in which the facility measurement value is measured via a network (not illustrated). For example, the correction value output to the terminal device is displayed on the screen of the terminal device. For example, a person who has confirmed the correction value displayed on the screen of the terminal device can recognize a difference between the facility measurement data and the daily measurement data. For example, the output unit 157 may be configured to output the correction value of the common measurement item to a display device (not illustrated) or an external system.

(Operation)

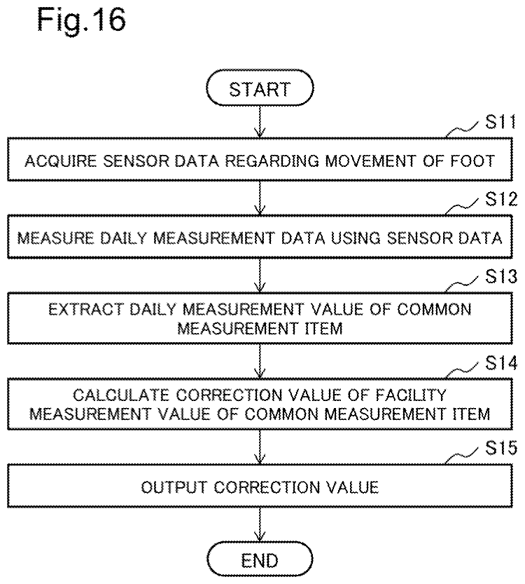

Next, an example of the operation of the information processing system 10 of the present example embodiment will be described with reference to the drawings. Here, an example of the operation of the biometric information processing device 15 of the information processing system 10 will be described with reference to a flowchart. FIG. 16 is a flowchart for explaining an example of the operation of the biometric information processing device 15. In the description along the flowchart of FIG. 16, the biometric information processing device 15 will be described as an operation subject.

In FIG. 16, first, the biometric information processing device 15 acquires sensor data regarding the movement of the foot from the measurement device 11 (step S11).

Next, the biometric information processing device 15 measures the daily measurement data using the acquired sensor data (step S12).

Next, the biometric information processing device 15 extracts a daily measurement value of the common measurement item from the measured daily measurement data (step S13).

Next, the biometric information processing device 15 calculates a correction value of the facility measurement value of the common measurement item based on the extracted daily measurement value of the common measurement item (step S14).

Next, the biometric information processing device 15 outputs the calculated correction value of the facility measurement value of the common measurement item (step S15). The correction value of the facility measurement value of the common measurement item output from the biometric information processing device 15 is used according to the application.

As described above, the information processing system of the present example embodiment includes the measurement device and the biometric information processing device. The measurement device is disposed on the user's footwear. The measurement device measures the spatial acceleration and the spatial angular speed according to the walking of the user. The measurement device generates sensor data based on the measured spatial acceleration and spatial angular speed. The measurement device outputs the generated sensor data to the biometric information processing device. The biometric information processing device includes a measurement unit, an extraction unit, a storage unit, a correction unit, and an output unit. The measurement unit receives, from the measurement device, sensor data related to the movement of the foot of the user. The measurement unit measures the daily measurement data using the received sensor data. The extraction unit extracts a daily measurement value of a common measurement item common to the facility measurement data measured in the facility from the daily measurement data measured by the measurement unit. The storage unit stores the facility measurement value of the common measurement item. The correction unit corrects the facility measurement value of the common measurement item stored in advance in the storage unit based on the daily measurement value of the common measurement item extracted by the extraction unit. The output unit outputs the facility measurement value corrected by the correction unit.

The information processing system of the present example embodiment corrects the facility measurement value of the common measurement item based on the daily measurement value of the common measurement item. Therefore, according to the present example embodiment, it is possible to correct a difference in measurement values (biometric data) for evaluating the exercise function, measured in different environments such as facilities and daily life.

In one aspect of the present example embodiment, the correction unit calculates a deviation between the representative value of the distribution of the daily measurement values of the common measurement item and the facility measurement value of the common measurement item. The correction unit corrects the facility measurement value of the common measurement item using the calculated deviation. According to the present aspect, since the facility measurement value can be corrected based on the representative value of the distribution of the daily measurement values accumulated in daily walking, it is possible to perform highly accurate correction reflecting more daily life.

### Second Example Embodiment

Next, an information processing system according to a second example embodiment will be described with reference to the drawings. The information processing system according to the present example embodiment calculates a correction value of a facility measurement value of a facility measurement item that is not included in daily measurement data based on a daily measurement value measured by a measurement device. Hereinafter, the facility measurement value of the facility measurement item that is not included in the daily measurement data is referred to as a facility measurement value of the facility measurement item.

(Configuration)

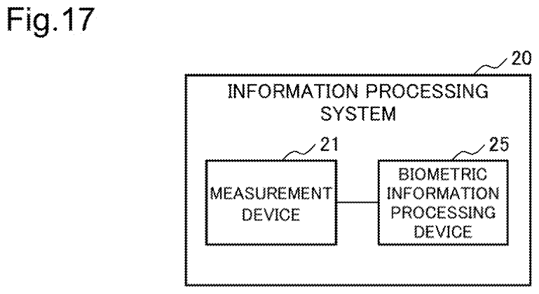

FIG. 17 is a block diagram illustrating an example of a configuration of an information processing system 20 according to the present example embodiment. The information processing system 20 includes a measurement device 21 and a biometric information processing device 25. The measurement device 21 and the biometric information processing device 25 may be connected by wire or wirelessly. The measurement device 21 and the biometric information processing device 25 may be configured by a single device. The information processing system 20 may include only the biometric information processing device 25 except for the measurement device 21 from the configuration of the information processing system 20.

The measurement device 21 is installed on the foot portion. The measurement device 21 has a configuration similar to that of the measurement device 11 of the first example embodiment. The measurement device 21 measures acceleration (also referred to as spatial acceleration) and angular speed (also referred to as spatial angular speed) as physical quantities related to the movement of the foot of the user wearing footwear such as shoes. The measurement device 21 converts the measured physical quantity into digital data (also referred to as sensor data). The measurement device 21 transmits the converted sensor data to the biometric information processing device 25.

The biometric information processing device 25 receives sensor data from the measurement device 21. The biometric information processing device 25 measures daily measurement data using the received sensor data. For example, the daily measurement data includes a stride length, a gait speed, a maximum dorsiflexion angle, a maximum plantarflexion angle, a foot elevation height, a diversion amount, and a foot angle. The biometric information processing device 25 extracts a common measurement item with the facility measurement data from among the measured daily measurement data. The biometric information processing device 25 calculates a correction value of the extracted common measurement item. The biometric information processing device 25 corrects the facility measurement value of the facility measurement item related to the common measurement item using the calculated correction value of the common measurement item.

For example, the biometric information processing device 25 measures a stride length, a gait speed, a maximum dorsiflexion angle, a maximum plantarflexion angle, a foot elevation height, a diversion amount, and a foot angle as daily measurement values. For example, the biometric information processing device 25 extracts daily measurement values of the stride length and the gait speed as common measurement items. The biometric information processing device 25 calculates correction values of the stride length and the gait speed. For example, the biometric information processing device 25 calculates the correction amount related to a knee joint load related to the stride length using the calculated correction value of the stride length. The knee joint load is a joint moment of the knee joint. The larger the value of the knee joint load, the larger the burden on the knee. For example, the biometric information processing device 25 calculates a correction value of gait stability related to the stride length and the gait speed using the calculated correction amounts of the stride length and the gait speed. The gait stability is the deviation of the center of gravity from the load bearing surface. The gait stability is more stable as it is closer to 0. When walking with the center of gravity eccentric forward, gait stability is positively inclined. When walking with the center of gravity eccentric backward, gait stability is negatively inclined. The facility measurement value related to the common measurement item is not limited to the knee joint load and gait stability. The facility measurement value related to the common measurement item may be appropriately selected according to the exercise function of the evaluation target.

The biometric information processing device 25 outputs a correction value of the facility measurement value of the facility measurement item related to the common measurement item. For example, the biometric information processing device 25 outputs the correction value of the facility measurement value of the facility measurement item corrected by the correction value. For example, the biometric information processing device 25 outputs the correction value to a terminal device browsable in the facility in which the facility measurement data is measured. For example, the correction value output to the terminal device is displayed on the screen of the terminal device. For example, a person who has confirmed the correction value displayed on the screen of the terminal device can recognize a difference between the facility measurement data and the daily measurement data. For example, the biometric information processing device 25 may be configured to output the correction value of the facility measurement item to a display device (not illustrated) or an external system.

(Biometric Information Processing Device)

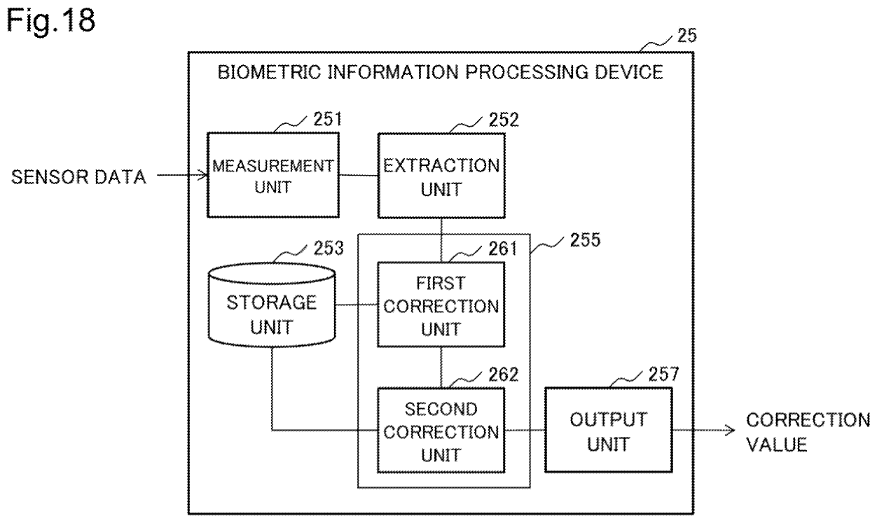

Next, details of the biometric information processing device 25 included in the information processing system 20 will be described with reference to the drawings. FIG. 18 is a block diagram illustrating an example of a configuration of the biometric information processing device 25. The biometric information processing device 25 includes a measurement unit 251, an extraction unit 252, a storage unit 253, a correction unit 255, and an output unit 257. The correction unit 255 includes a first correction unit 261 and a second correction unit 262.

The measurement unit 251 acquires sensor data from the measurement device 21 installed on the footwear worn by the user. The measurement unit 251 has the same configuration as the measurement unit 151 of the first example embodiment. The measurement unit 251 measures daily measurement data using the acquired sensor data. For example, the measurement unit 251 measures daily measurement data such as a stride length, a gait speed, a maximum dorsiflexion angle, a maximum plantarflexion angle, a foot elevation height, a diversion amount, and a foot angle.

The extraction unit 252 extracts a daily measurement value of a common measurement item with the facility measurement data from the daily measurement data measured by the measurement unit 251. For example, the extraction unit 252 extracts a representative value of the daily measurement value of the common measurement item with the facility measurement data from the daily measurement data continuously measured by the measurement unit 251. For example, the extraction unit 252 extracts the stride length and the gait speed as common measurement items.

The storage unit 253 stores common measurement items. The storage unit 253 stores the facility measurement value of the facility measurement item related to the common measurement item. For example, the storage unit 253 stores the facility measurement values of the stride length and the gait speed. For example, the storage unit 253 stores the facility measurement values of the knee joint load and the gait stability, which are facility measurement items related to the stride length and the gait speed.

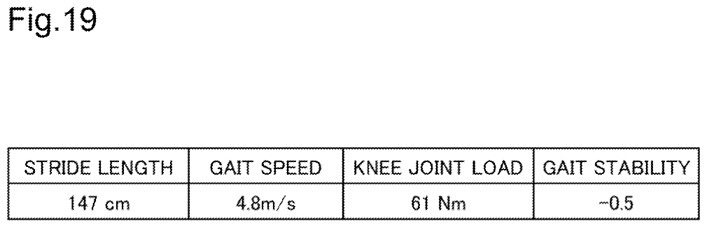

FIG. 19 is an example of the facility measurement value stored in the storage unit 253. In the example of FIG. 19, a facility measurement value 147 cm of the stride length, a facility measurement value 4.8 m/s of the gait speed, a facility measurement value 61 Nm (Newton meter) of the knee joint load, and a facility measurement value −0.5 of the gait stability are stored in the storage unit 153. The knee joint load and the gait stability are measured by a measurement method different from the measurement device 21 that measures the daily measurement values of the stride length and the gait speed. For example, a knee joint load is measured using motion capture and a floor reaction force meter. For example, the gait stability is observed as a response to disturbance stimuli on a treadmill. The facility measurement value stored in the storage unit 253 can be updated at an arbitrary timing. For example, the facility measurement value stored in the storage unit 253 is updated according to data transmitted from a facility such as a sports gym or a hospital via a network (not illustrated). A method for updating the facility measurement value stored in the storage unit 253 is not particularly limited.

The first correction unit 261 calculates a correction value of the daily measurement value of the common measurement item and the facility measurement value. The first correction unit 261 has the same configuration as the correction unit 155 of the first example embodiment. For example, the first correction unit 261 calculates correction values of the stride length and the gait speed. For example, the first correction unit 261 calculates a difference between the daily measurement value and the facility measurement value of the common measurement item as the correction value. For example, the first correction unit 261 calculates a deviation between the representative value of the distribution of the daily measurement values of the common measurement item and the facility measurement value as the correction value. For example, as the representative value of the distribution of the daily measurement values of the common measurement item, the first correction unit 261 uses, as the representative value, an average value such as an arithmetic mean, a geometric mean, or a harmonic mean of a plurality of daily measurement values constituting the distribution. For example, the first correction unit 261 uses, as a representative value of the distribution of the daily measurement values of the common measurement item, a mode value or a median value of a plurality of daily measurement values constituting the distribution.

The second correction unit 262 corrects the facility measurement value of the facility measurement item related to the common measurement item using the correction value of the common measurement item calculated by the first correction unit 261. For example, the second correction unit 262 calculates the correction amount regarding the knee joint load related to the stride length using the correction value of the stride length. For example, the second correction unit 262 calculates the correction amount regarding the gait stability related to the stride length and the gait speed using the correction values of the stride length and the gait speed. The second correction unit 262 calculates the correction values of the knee joint load and the gait stability using the calculated correction amount.

For example, the second correction unit 262 calculates the correction amount of the knee joint load using the method of NPL 1 (NPL 1: Lara Allet, et al., “The influence of stride-length on plantar foot-pressures and joint moments”, Gait & Posture 34, (2011), pp. 300-306). According to NPL 1, when the stride length increases by 20% (percent), the knee joint load increases by 18%. Here, regarding the stride length, it is assumed that the daily measurement value is 147 cm and the facility measurement value is 140 cm. In this case, the correction amount C_{KJL} of the knee joint load is calculated using the following Expression 1.

$${C}_{KJL} = \frac{140 - 147}{147} \times \frac{18}{20} = - 4.8 \qquad ( 1 )$$

That is, when the knee joint load before correction stored in the storage unit 253 is 61 Nm, the knee joint load after correction has a value (58 Nm) reduced by the correction amount C_{KJL} (−4.8%) calculated using the above Expression 1.

For example, the second correction unit 262 calculates the correction amount of the gait stability using the method of NPL 2 (NPL 2: D. D. Espy, et al., “Independent Influence of Gait Speed and Step Length on Stability and Fall Risk”, Gait Posture, 2010 July, 32(3), 378-82). According to NPL 2, when the normalized gait speed is increased by 1, the gait stability is improved by 0.229. The normalized gait speed is a value obtained by dividing the gait speed by a square root of a value obtained by multiplying the height by 9.8. According to NPL 2, when the normalized stride increases by 1, the gait stability deteriorates by 0.901. The normalized stride is a value obtained by dividing the stride (½ of the stride length) by the height. Here, regarding the stride length and the gait speed of the subject having a height of 1.84 m, it is assumed that the daily measurement values are 147 cm and 4.5 m/s, and the facility measurement values are 140 cm and 4.8 m/s. In this case, the correction amount C_{WS} of the gait stability is calculated using the following Expression 2.

$${C}_{WS} = 0.229 \times \frac{- 0.3}{\sqrt{9.8 \times 1.84}} - 0.901 \times \frac{- 0.7}{2 \times 1.84} = 0.155 \qquad ( 2 )$$

That is, when the gait stability before correction stored in the storage unit 253 is −0.5, the gait stability after correction is a value (−0.345) improved by the correction amount C_{WS} (0.155) calculated using the above Expression 2.

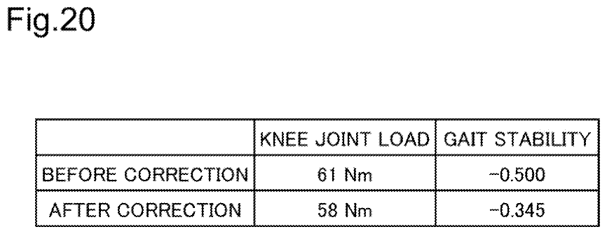

FIG. 20 is a table summarizing values of the facility measurement items before and after correction by the first correction unit 261 and the second correction unit 262. In the table of FIG. 20, a row of facility measurement values of the knee joint load and the gait stability before correction and a row of correction values of the knee joint load and the gait stability after correction are vertically arranged. For example, the trainer who instructs the exercise to the user who is the acquisition source of the data of FIG. 20 can review the training menu created based on the data before correction by referring to the knee joint load and gait stability after correction. Compared with the stride length and the gait speed, the knee joint load and the gait stability are clear as indices for achieving the exercise function. Therefore, based on the knee joint load and gait stability, a more accurate training menu can be created.

The output unit 257 outputs the correction values of the common measurement item and the facility measurement item. For example, the output unit 257 outputs the values of the common measurement item and the facility measurement item corrected by the correction value. For example, the output unit 257 displays the values of the common measurement item and the facility measurement item corrected by the correction value on the screen of the mobile terminal carried by the user. For example, the output unit 257 outputs the correction value to a terminal device browsable in the facility in which the facility measurement value is measured. For example, the correction value output to the terminal device is displayed on the screen of the terminal device. For example, a person who has confirmed the correction value displayed on the screen of the terminal device can recognize the correction value of the facility measurement item reflecting the daily measurement value. For example, the output unit 257 may be configured to output the correction value of the common measurement item to a display device (not illustrated) or an external system.

(Operation)

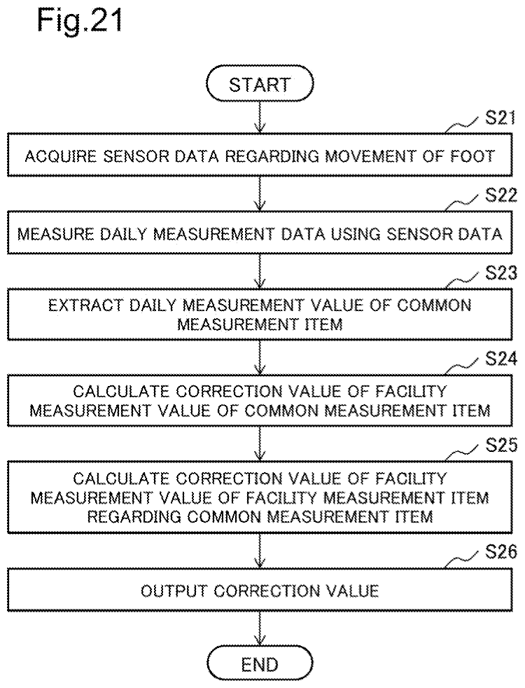

Next, an example of the operation of the information processing system 20 of the present example embodiment will be described with reference to the drawings. Here, an example of the operation of the biometric information processing device 25 of the information processing system 20 will be described with reference to a flowchart. FIG. 21 is a flowchart for explaining an example of the operation of the biometric information processing device 25. In the description along the flowchart of FIG. 21, the biometric information processing device 25 will be described as an operation subject.

In FIG. 21, first, the biometric information processing device 25 acquires sensor data regarding the movement of the foot from the measurement device 21 (step S21).

Next, the biometric information processing device 25 measures the daily measurement data using the acquired sensor data (step S22).

Next, the biometric information processing device 25 extracts the daily measurement value of the common measurement item from the measured daily measurement data (step S23).

Next, the biometric information processing device 25 calculates a correction value of the facility measurement value of the common measurement item based on the extracted daily measurement value of the common measurement item (step S24).

Next, the biometric information processing device 25 calculates a correction value of the facility measurement value of the facility measurement item related to the common related item based on the calculated correction value of the facility measurement value of the common measurement item (step S25).

Next, the biometric information processing device 25 outputs the calculated correction value of the facility measurement value of the facility measurement item (step S26). The correction value of the facility measurement value of the facility measurement item output from the biometric information processing device 25 is used according to the application.

### Application Example

Next, an application example of the present example embodiment will be described with reference to the drawings. In the following application example, an example in which sensor data measured by the measurement device 21 installed on the shoe worn by the user is processed by an application installed in the mobile terminal of the user will be described. In the following application example, it is assumed that an application that exhibits the function of the biometric information processing device 25 of the second example embodiment is installed in a mobile terminal.

### Application Example 1

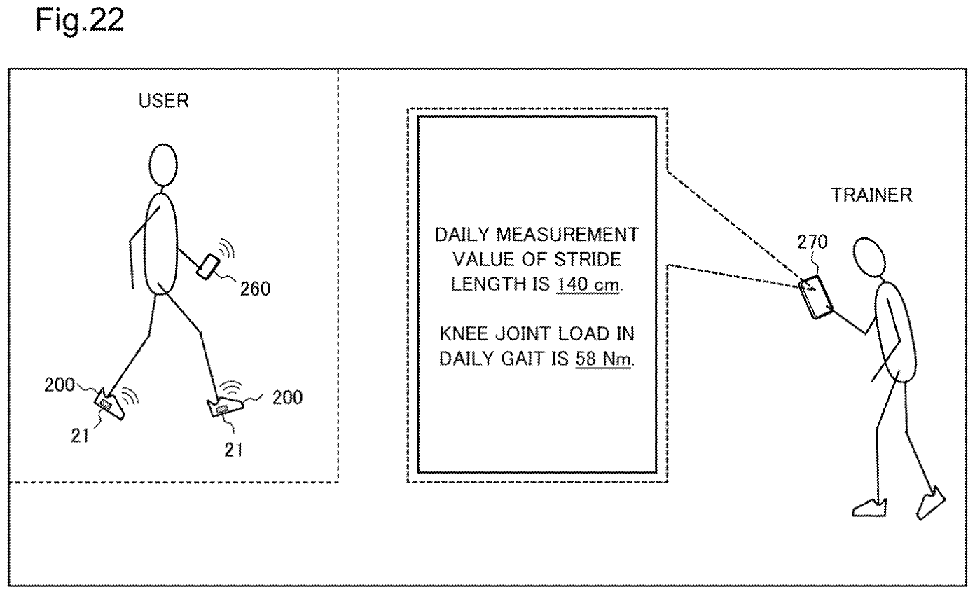

FIG. 22 is a conceptual diagram for explaining Application Example 1. In the present application example, the sensor data is transmitted to a mobile terminal 260 carried by the user according to the walking of the user wearing the shoe 200 on which the measurement device 21 is installed. The application (biometric information processing device 25) installed in the mobile terminal 260 outputs a correction value for correcting the facility measurement value of the facility measurement item related to the common measurement item with the facility measurement data based on the received sensor data. The correction value output from the application is transmitted from the mobile terminal 260 of the user to a mobile terminal 270 of the trainer who manages the exercise of the user.

The information regarding the correction value transmitted to the mobile terminal 270 of the trainer is displayed on the screen of the mobile terminal 270. For example, when the trainer touches a button displayed on the screen of the mobile terminal 270, information regarding the corrected facility measurement data is displayed on the screen of the mobile terminal 270. In the example of FIG. 22, information “The daily measurement value of the stride length is 140 cm. The knee joint load in a daily walking is 58 Nm.” is displayed. For example, the trainer who has seen the information displayed on the screen of the mobile terminal 270 can create a training menu relevant to the information.

The facility measurement value is not limited to a measurement value measured using a specialized instrument for evaluating an exercise function. For example, the facility measurement value may be generated visually by a trainer of a sports gym or an expert such as a physical therapist. For example, the correction value calculated by the biometric information processing device 25 may be reflected in the training menu created by the trainer. For example, in a case where daily measurement data related to an index value of an exercise function of a user visually determined by a trainer in a facility can be measured as a common measurement item, the index value may be corrected based on the common measurement item. For example, the index value corrected based on the common measurement item is displayed on the screen of the mobile terminal 270 carried by the trainer. For example, the trainer who sees the information displayed on the screen of the mobile terminal 270 can create a training menu relevant to the index value. For example, the training menu may be customized based on methods specific to the training gym or trainer.

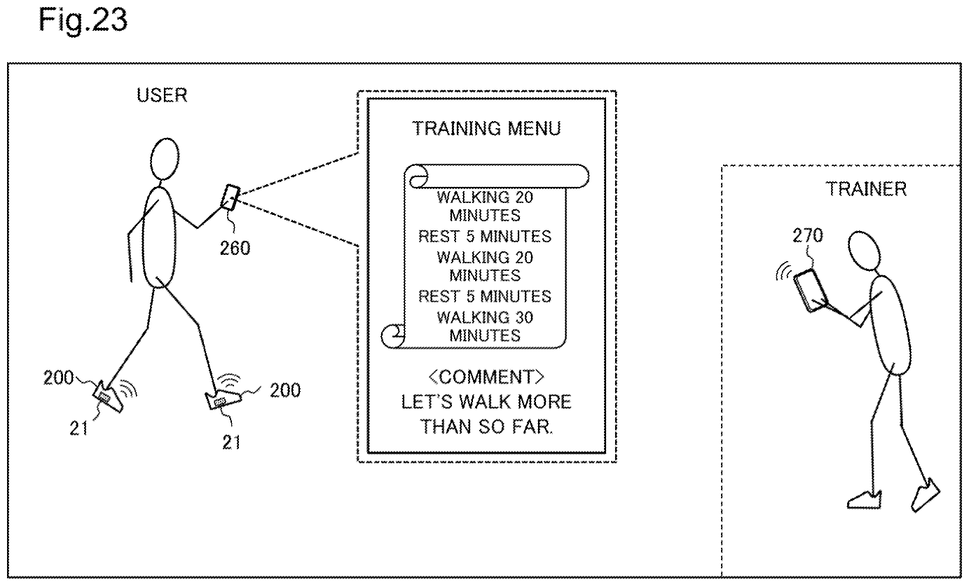

FIG. 23 is an example of displaying the training menu created in the example of FIG. 22 on the screen of the mobile terminal 260 carried by the user. For example, the training menu created by the trainer who has viewed the information displayed on the screen of the mobile terminal 270 is transmitted to the mobile terminal 260 of the user to be managed together with the comment of the trainer. The information regarding the training menu transmitted to the mobile terminal 260 of the user is displayed on the screen of the mobile terminal 260. For example, the user who has seen the information displayed on the screen of the mobile terminal 260 can exercise according to the training menu or the comment.

In the present application example, the training menu dedicated to the user created based on the facility measurement value is corrected based on the daily measurement data of the user. Therefore, according to the present application example, it is possible to create a training menu reflecting the original exercise function of the user.

### Application Example 2

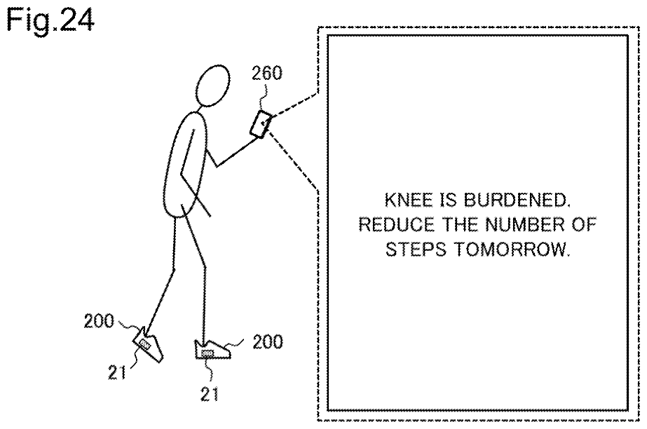

FIG. 24 is a conceptual diagram for explaining Application Example 2. In the present application example, the sensor data is transmitted to a mobile terminal 260 carried by the user according to the walking of the user wearing the shoe 200 on which the measurement device 21 is installed. The application (biometric information processing device 25) installed in the mobile terminal 260 calculates a correction value for correcting the facility measurement value of the facility measurement item related to the common measurement item with the facility measurement data based on the received sensor data.

For example, the application verifies the degree of difference from the facility measurement value based on the calculated correction value. For example, in a case where the correction value of the facility measurement value corrected based on the daily measurement value excessively exceeds the original facility measurement value, the application generates recommendation information recommending reduction in the number of steps. For example, in a case where the correction value of the facility measurement value corrected based on the daily measurement value is excessively lower than the original facility measurement value, the application generates recommendation information recommending an increase in the number of steps. For example, in a case where the correction value of the facility measurement value corrected based on the daily measurement value is close to the original facility measurement value, the application generates recommendation information recommending maintaining the number of steps at that time. For example, the application displays recommendation information relevant to the calculated correction value on the screen of the mobile terminal 260. In the example of FIG. 24, in response to the correction value of the facility measurement value excessively exceeding the original facility measurement value, recommendation information of “Knee is burdened. Reduce the number of steps tomorrow.” is displayed on the screen of the mobile terminal 260. For example, the user who has seen the information displayed on the screen of the mobile terminal 260 can review daily walking according to the information.

For example, the application compares an integrated value of the knee joint load in a predetermined period (1 hour, 1 day, etc.) with a predetermined threshold based on the calculated correction value, and notifies the mobile terminal 260 of advice according to the knee joint load of the user. For example, in a case where the integrated value in the predetermined period of the correction value of the facility measurement value of the knee joint load corrected based on the daily measurement value exceeds a predetermined threshold, the application generates recommendation information recommending reduction of the number of steps. The predetermined threshold in this case is a limit value of the knee joint load for not damaging the knee joint. For example, in a case where the integrated value in a predetermined period of the correction value of the facility measurement value of the knee joint load corrected based on the daily measurement value is close to a predetermined threshold, the application generates recommendation information recommending maintaining the number of steps. The predetermined threshold in this case is an appropriate value of the knee joint load necessary and sufficient for maintaining health. For example, the application displays recommendation information relevant to the calculated correction value on the screen of the mobile terminal 260. In the example of FIG. 24, in response to the integrated value in the predetermined period of the correction value of the facility measurement value of the knee joint load corrected based on the daily measurement value exceeding the predetermined threshold, recommendation information of “Knee is burdened. Reduce the number of steps tomorrow.” is displayed on the screen of the mobile terminal 260. For example, the user who has seen the information displayed on the screen of the mobile terminal 260 can review daily walking according to the information.

In the present application example, the recommendation information according to the relationship between the correction value of the facility measurement value and the original facility measurement value or the relationship between the integrated value of the correction value of the facility measurement value corrected based on the daily measurement value in the predetermined period and the predetermined threshold is displayed on the screen of the mobile terminal 260 carried by the user. Therefore, according to the present application example, the recommendation information reflecting the user's original exercise function can be provided to the user. For example, for an elderly person who is concerned about the pain of the knee but is desired to continue walking for health, it is desirable to walk with an appropriate number of steps in daily life. According to the present application example, it is recommended to reduce the number of steps in a case where the number of steps is too large, and it is recommended to increase the number of steps in a case where the number of steps is too small according to the actual situation of the knee joint load of the user, so that the user can continue appropriate walking.

As described above, the information processing system of the present example embodiment includes the measurement device and the biometric information processing device. The measurement device is disposed on the user's footwear. The measurement device measures the spatial acceleration and the spatial angular speed according to the walking of the user. The measurement device generates sensor data based on the measured spatial acceleration and spatial angular speed. The measurement device outputs the generated sensor data to the biometric information processing device. The biometric information processing device includes a measurement unit, an extraction unit, a storage unit, a correction unit, and an output unit. The measurement unit receives, from the measurement device, sensor data related to the movement of the foot of the user. The measurement unit measures the daily measurement data using the received sensor data. The extraction unit extracts a daily measurement value of a common measurement item common to the facility measurement data measured in the facility from the daily measurement data measured by the measurement unit. The storage unit stores the facility measurement value of the common measurement item and the facility measurement value of the facility measurement item related to the common measurement item. The correction unit includes a first correction unit and a second correction unit.

The first correction unit corrects the facility measurement value of the common measurement item based on the daily measurement value of the common measurement item extracted by the extraction unit. The second correction unit corrects the facility measurement value of the facility measurement item related to the common measurement item based on the facility measurement value of the common measurement item corrected by the first correction unit. The output unit outputs the facility measurement value of the facility measurement item corrected by the second correction unit.

The information processing system of the present example embodiment corrects the facility measurement value of the facility measurement item related to the common measurement item based on the daily measurement value of the common measurement item. Therefore, according to the present example embodiment, it is possible to correct a difference between measurement values (biometric data) of measurement items related to measurement items measured in different environments such as facilities and daily life.

In one aspect of the present example embodiment, the extraction unit extracts the daily measurement value of the stride length as the common measurement item. The correction unit corrects the facility measurement value of the knee joint load, which is the facility measurement item related to the stride length, based on the daily measurement value of the stride length extracted by the extraction unit. According to the present aspect, the facility measurement value of the knee joint load measured in an environment (facility) different from daily life can be corrected based on the stride length measured in daily life.

In one aspect of the present example embodiment, the extraction unit extracts the daily measurement values of the stride length and the gait speed as the common measurement items. The correction unit corrects a facility measurement value of gait stability, which is a facility measurement item related to the stride length and the gait speed, based on the daily measurement values of the stride length and the gait speed extracted by the extraction unit. According to the present aspect, the facility measurement value of the gait stability measured in an environment (facility) different from daily life can be corrected based on the stride length and the gait speed measured in daily life.

In one aspect of the present example embodiment, the output unit outputs the correction value of the facility measurement value to a terminal device that can be browsed by a trainer who manages exercise of the user. The output unit acquires the training menu created by the trainer according to the correction value of the facility measurement value. For example, the output unit acquires the training menu via an input unit (not illustrated). The output unit displays the acquired training menu on a screen of a terminal device that can be browsed by the user. According to the present aspect, it is possible to provide the training menu updated by the trainer to the user based on the facility measurement value reflecting the daily measurement value.

In one aspect of the present example embodiment, the output unit displays the recommendation information according to the correction value of the facility measurement value on a screen of a terminal device that can be browsed by the user. According to the present aspect, it is possible to provide the user with recommendation information reflecting a daily exercise situation.

### Third Example Embodiment

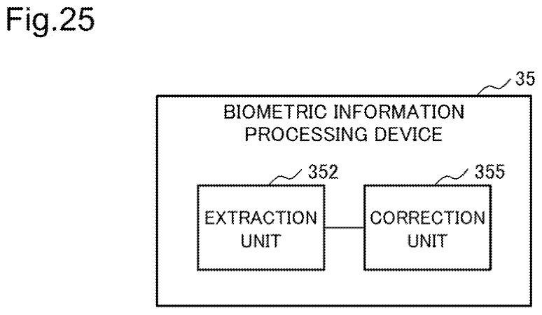

Next, a biometric information processing device according to a third example embodiment will be described with reference to the drawings. The biometric information processing device of the present example embodiment has a simplified configuration of the biometric information processing devices of the first and the second example embodiments. FIG. 25 is a block diagram illustrating an example of a configuration of a biometric information processing device 35 according to the present example embodiment. The biometric information processing device 35 includes an extraction unit 352 and a correction unit 355.

The extraction unit 352 extracts a daily measurement value of a common measurement item common to the facility measurement data measured in the facility from the daily measurement data measured using the sensor data regarding the movement of the foot of the user. The correction unit 355 corrects the facility measurement value of the common measurement item stored in advance based on the daily measurement value of the common measurement item extracted by the extraction unit 352.

The biometric information processing device according to the present example embodiment corrects the facility measurement value of the common measurement item based on the daily measurement value of the common measurement item. Therefore, according to the present example embodiment, it is possible to correct a difference in measurement values (biometric data) for evaluating the exercise function, measured in different environments such as facilities and daily life.

(Hardware)

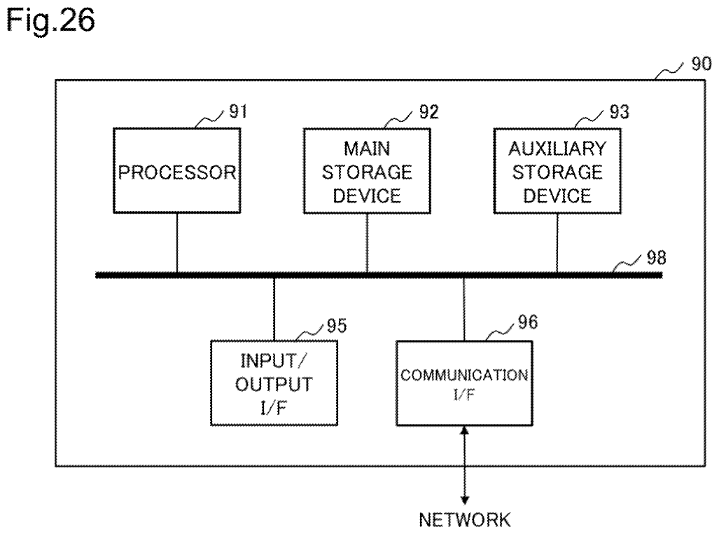

Here, a hardware configuration for executing processing of the control unit according to each example embodiment of the present disclosure will be described using an information processing device 90 of FIG. 26 as an example. The information processing device 90 in FIG. 26 is a configuration example for executing the control and processing of each example embodiment, and does not limit the scope of the present disclosure.

As illustrated in FIG. 26, the information processing device 90 includes a processor 91, a main storage device 92, an auxiliary storage device 93, an input/output interface 95, and a communication interface 96. In FIG. 26, the interface is abbreviated as an I/F. The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program stored in the auxiliary storage device 93 or the like in the main storage device 92. The processor 91 executes the program developed in the main storage device 92. In the present example embodiment, a software program installed in the information processing device 90 may be used. The processor 91 executes processing or control according to the present example embodiment.

The main storage device 92 has an area in which a program is developed. A program stored in the auxiliary storage device 93 or the like is developed in the main storage device 92 by the processor 91. The main storage device 92 is implemented by, for example, a volatile memory such as a dynamic random access memory (DRAM). A nonvolatile memory such as a magnetoresistive random access memory (MRAM) may be configured and added as the main storage device 92.

The auxiliary storage device 93 stores various data such as programs. The auxiliary storage device 93 is implemented by a local disk such as a hard disk or a flash memory. Various data may be stored in the main storage device 92, and the auxiliary storage device 93 may be omitted.

The input/output interface 95 is an interface for connecting the information processing device 90 and a peripheral device. The communication interface 96 is an interface for connecting to an external system or device through a network such as the Internet or an intranet based on a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input information and settings. When the touch panel is used as an input device, the display screen of the display device may also serve as the interface of the input device. Data communication between the processor 91 and the input device may be mediated by the input/output interface 95.

The information processing device 90 may be provided with a display device for displaying information. In a case where a display device is provided, the information processing device 90 preferably includes a display control device (not illustrated) for controlling display of the display device. The display device may be connected to the information processing device 90 via the input/output interface 95.

The information processing device 90 may be provided with a drive device. The drive device mediates reading of data and a program from a recording medium, writing of a processing result of the information processing device 90 to the recording medium, and the like between the processor 91 and the recording medium (program recording medium). The drive device may be connected to the information processing device 90 via the input/output interface 95.

The above is an example of the hardware configuration for enabling the control and processing according to each example embodiment of the present invention. The hardware configuration of FIG. 26 is an example of a hardware configuration for executing the control and processing of each example embodiment, and does not limit the scope of the present invention. A program for causing a computer to execute the control and processing according to each example embodiment is also included in the scope of the present invention. Further, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be implemented by a semiconductor recording medium such as a universal serial bus (USB) memory or a secure digital (SD) card. The recording medium may be implemented by a magnetic recording medium such as a flexible disk, or another recording medium. When a program executed by the processor is recorded in a recording medium, the recording medium is associated to a program recording medium.

The components of each example embodiment may be arbitrarily combined. The components of each example embodiment may be implemented by software or may be implemented by a circuit. For example, the measurement device of each example embodiment is achieved by a microcomputer, a microcontroller, or the like. For example, the biometric information processing device of each example embodiment is achieved by a function of a computer included in a cloud or a server. The biometric information processing device of each example embodiment may be achieved by software installed in a smartphone, a tablet, a notebook type or an installation type personal computer, or the like.

Although the present invention has been described with reference to the example embodiments, the present invention is not limited to the above example embodiments. Various modifications that can be understood by those skilled in the art can be made to the configuration and details of the present invention within the scope of the present invention.

### REFERENCE SIGNS LIST

10, 20 information processing system
11, 21 measurement device
15, 25, 35 biometric information processing device
111 acceleration sensor
112 angular speed sensor
113 control unit
115 transmission unit
151, 251 measurement unit
152, 252, 352 extraction unit
153, 253 storage unit
155, 255, 355 correction unit
157, 257 output unit
261 first correction unit
262 second correction unit

What is claimed is:

1. A biometric information processing device comprising:
a memory storing instructions; and
a processor connected to the memory and configured to execute the instructions to:
extract a daily measurement value of a common measurement item common to facility measurement data measured in a facility from daily measurement data measured using a sensor data regarding movement of a foot of a user; and
correct a facility measurement value of the common measurement item stored in advance based on the extracted daily measurement value of the common measurement item.

2. The biometric information processing device according to claim 1, wherein
the processor is configured to execute the instructions to
calculate a deviation between a representative value of a distribution of daily measurement values of the common measurement item and a facility measurement value of the common measurement item, and
correct a facility measurement value of the common measurement item using the calculated deviation.

3. The biometric information processing device according to claim 1, wherein
the processor is configured to execute the instructions to
correct a facility measurement value of the common measurement item based on a daily measurement value of the common measurement item, and
correct a facility measurement value of a facility measurement item related to the common measurement item based on the corrected facility measurement value of the common measurement item.

4. The biometric information processing device according to claim 3, wherein
the processor is configured to execute the instructions to
extract a daily measurement value of a stride length as the common measurement item, and
correct a facility measurement value of a knee joint load that is a facility measurement item related to the stride length based on the extracted daily measurement value of the stride length.

5. The biometric information processing device according to claim 3, wherein
the processor is configured to execute the instructions to
extract daily measurement values of a stride length and a gait speed as the common measurement items, and
correct a facility measurement value of gait stability, which is a facility measurement item related to the stride length and the gait speed, based on the extracted daily measurement values of the stride length and the gait speed.

6. The biometric information processing device according to claim 1, wherein
the processor is configured to execute the instructions to
output a correction value of the facility measurement value to a terminal device browsable by a trainer who manages exercise of the user,
acquire a training menu created by the trainer according to a correction value of the facility measurement value, and
display the acquired training menu on a screen of a terminal device browsable by the user.

7. The biometric information processing device according to claim 1, wherein
the processor is configured to execute the instructions to
display recommendation information according to a correction value of the facility measurement value on a screen of a terminal device browsable by the user.

8. An information processing system comprising:
a biometric information processing device according to claim 1; and
a measurement device that is disposed on footwear of a user, measures a spatial acceleration and a spatial angular speed according to walking of the user, generates sensor data based on the measured spatial acceleration and spatial angular speed, and outputs the generated sensor data to the biometric information processing device.

9. A biometric information processing method causing a computer to execute:
extracting a daily measurement value of a common measurement item common to facility measurement data measured in a facility from daily measurement data measured using the sensor data regarding movement of a foot of a user; and
correcting a facility measurement value of the common measurement item stored in advance based on the extracted daily measurement value of the common measurement item.

10. A non-transitory recording medium having stored therein a program causing a computer to execute:
extracting a daily measurement value of a common measurement item common to facility measurement data measured in a facility from daily measurement data measured using the sensor data regarding movement of a foot of a user; and
correcting a facility measurement value of the common measurement item stored in advance based on the extracted daily measurement value of the common measurement item.
